(19) United States
(12) Patent Application Publication
(10) Pub. No.: US 2026/0294476 A1
(43) Pub. Date: Oct. 1, 2026

(54) DEVICES AND METHODS FOR TENSION PNEUMOTHORAX TREATMENT

(71) Applicant: MEDPACK Devices L.L.C., Raleigh, NC (US)

(72) Inventors: Dante Tomaino, Raleigh, NC (US); Aakarsh Gundu, Apex, NC (US); Rachel Sides, Concord, NC (US); Riley Mann, Clayton, NC (US); Rebecca Pope, Kenly, NC (US)

(21) Appl. No.: 19/095,389

(22) Filed: Mar. 31, 2025

(51) Int. Cl. A61B 17/34; A61M 25/00; A61M 25/06; A61M 39/24

(52) U.S. Cl. CPC A61B 17/3474; A61M 25/0606; A61M 39/24; A61M 2025/0063; A61M 2039/2406; A61M 2205/073; A61M 2210/101

(57) ABSTRACT

Methods and devices for treating a tension pneumothorax. The tension pneumothorax treatment methods and devices prevent kinking or dislodgement, reduce the probability of accidental needlesticks, and allow unidirectional fluid flow from a chest cavity to prevent air ingress. The pneumothorax treatment device can include an adjustable housing, a needle, a one-way valve, and a hub.

### BACKGROUND

When functioning properly, the lungs act as a thin spongy organ adhered to the chest cavity through negative pleural pressure. During respiration, the pleural cavity between the lungs and the chest cavity always remains below atmospheric pressure despite the intrapulmonary pressure fluctuating pulling air into the lungs and pushing it back outward. A positive pressure within the pleural space would cause the lungs to become freed from the chest cavity, thus reducing respiration while also decreasing venous return in the heart.

A pneumothorax, whether caused through disease, injury, or spontaneous occurrence, is the presence of air within the pleural cavity causing the lung to either partially or completely collapse. Typically, the pleural cavity is inflated, which in turn compresses the lungs and prevents normal respiration. The pleural cavity is positioned within a patient's chest wall and maintains negative pressure relative to atmosphere during respiration to induce elastic recoil of the lungs. The pressure difference between the pleural cavity and pulmonary alveoli allow the lungs to properly inflate.

A more severe case of a pneumothorax is the tension pneumothorax, often caused by a penetrating chest injury. Tension pneumothorax involves rupture of the pleural alveoli and allows airflow into the pleural cavity during inhalation. This significantly increases the pleural pressure and can cause complete collapse of a corresponding lung. A tension pneumothorax must be treated immediately as the accumulating air within the pleural space impairs ventilation and eventually leads to circulatory collapse.

When treating a tension pneumothorax, emergency medical providers will often perform a needle thoracostomy. This procedure typically includes the insertion of a 3.25-inch-long needle encompassed within a 14-gauge catheter into the pleural space to relieve air entrapped outside of the lungs. The tip of the needle is first inserted into the patient's chest wall, typically in the second intercostal space at the midclavicular line, or the fourth or fifth intercostal space at the midaxillary line. Once inserted to a depth reaching the pleural space, the catheter is slid over and off of the needle while removing the needle from the chest. The catheter, once placed, can relieve the pressure of entrapped air.

Some limitations of the current devices and methods leave medical providers exposed to the sharp needle after placing the catheter, an easily kinked or unsecured catheter hub, and an unprotected opening for potential fluid, bacteria, or air to return to the pleural space. These limitations mean that generally, a chest tube is placed in the patient in the emergency department even if the pneumothorax was initially managed successfully in the field. Current designs fail to address all of these problems in a unitary solution.

### FIELD

The present disclosure is generally directed to devices for treating pneumothoraces, more specifically treatment devices for tension pneumothorax.

### BRIEF SUMMARY OF THE DISCLOSURE

In some embodiments, a pneumothorax treatment device addressing the problems with current devices and systems is disclosed. The pneumothorax treatment device can include (1) a one-way valve to prevent air ingress into the pleural space, (2) a sharps-safety system for reducing the chance of accidental needlesticks, and (3) a supportive adhesive component to maintain positioning of the pneumothorax treatment device in a patient's chest well and to prevent kinking or dislodgement. The pneumothorax treatment device includes a sliding cover system designed to conceal the needle following device placement, thereby reducing the exposure of medical providers to the used needle point. Additionally, the pneumothorax treatment device includes a hub with a one-way valve designed to prevent the ability of air to re-enter the chest cavity of a patient. Further, in some embodiments, the pneumothorax treatment device may include an adhesive surface on the hub which could be used to better retain the device against the patient and reduce the chance of kinking or dislodgement.

In some embodiments, a device for removal of gas and/or fluid from a thoracic cavity of a patient is disclosed. The removal device includes a needle, tubing, a hub, a valve, and a safety mechanism. The needle includes a sharp distal end and a hollow body that allows fluid or gas to pass through. The tubing is positioned to surround the needle and includes one or more openings in a distal end of a catheter. The hub is attached to a proximal end of a catheter and includes the valve. The valve is operable to control the flow of gas and/or fluid. The safety mechanism is designed to conceal a distal tip of the needle within a housing when deployed. The needle and catheter is stored in the housing when in the undeployed state.

In some embodiments, the safety mechanism includes a movable component and a stationary component. The stationary component is attached (e.g., affixed) to a proximal end of the needle. In some embodiments, the movable component includes a body positioned within the stationary component while surrounding the needle. The movable component is operable to slide distally. In some embodiments, the safety mechanism further includes at least one latch. The at least one latch is operable to engage and retain the movable component in the distal position so the tip of the needle is concealed.

In some embodiments, a one-way valve is positioned within the hub. The one-way valve is in the open position when the needle is positioned within the catheter and passing through the valve. The one-way valve is in the closed position when the needle is removed from the catheter and hub. The one-way valve enables flow of fluid from the distal end of the catheter toward the proximal end of the hub but does not permit flow from the proximal end of the hub toward the distal end of the catheter.

In some embodiments, the hub includes a flat portion surrounding the attachment of the catheter to said hub. The flat portion includes an adhesive-coated surface, designed that when the catheter passes through the chest wall of a patient, the adhesive retains the hub against the chest wall and the flat portion of the hub provides additional stability. In some embodiments, the catheter includes a plurality of distal openings. Advantageously, this ensures that a single obstruction at one opening may not prevent flow through the cannula.

In at least one aspect of the present disclosure, a pneumothorax treatment device is disclosed. The pneumothorax treatment device can include a needle including a distal end, a hub including a valve, and a telescopic housing. The hub is connected to a proximal end of at least one tube. The telescopic housing can move between a deployed position and an undeployed position. In the deployed position, the distal end of the needle is concealed within the housing. In the undeployed position, the needle is unconcealed.

In at least one aspect of the present disclosure, the telescopic housing includes a stationary member and a movable member. The stationary member can be affixed to the proximal end of the needle. The needle can be contained within the stationary member.

In at least one aspect of the present disclosure, the movable member of the telescopic member surrounds the stationary member. The movable member is operable to slide distally from the stationary member.

In at least one aspect of the present disclosure, the telescopic housing can further include at least one latch. When in the deployed position, the at least one latch can engage and retain a corresponding fastener to conceal the needle.

In at least one aspect of the present disclosure, the valve of the pneumothorax treatment device is a one-way valve. The one-way valve is positioned to allow a flow of fluid from the distal end of the at least one tube toward the proximal end of the hub. Flow is not permitted from the proximal end of the hub toward the distal end of the at least one tube.

In at least one aspect of the present disclosure, the hub includes a flat exterior portion. The flat exterior portion is positioned near an attachment of the at least one tube to the hub. In at least one aspect of the present disclosure, the fat exterior portion includes an adhesive.

In at least one aspect of the present disclosure, the pneumothorax treatment device further includes a grip tab. The grip tab is positioned between the hub and the telescopic housing.

In at least one aspect of the present disclosure, a pneumothorax treatment device is disclosed. The pneumothorax treatment device includes a needle including a distal end, a hub including a valve, and a telescopic housing. The telescopic housing surrounds at least a portion of the needle. The telescopic housing is connected to a proximal end of the hub. The hub is further connected to at least one tube. The telescopic housing can move between a deployed position and an undeployed position. In the deployed position, the distal end of the needle is concealed within the housing. In the undeployed position, the needle is concealed.

In at least one aspect of the present disclosure, the telescopic housing can further include at least one latch. In the deployed position, the at least one latch can engage and retain a corresponding fastener to conceal the needle.

In at least one aspect of the present disclosure, the telescopic housing includes a stationary member and a movable member. The stationary member is affixed to the proximal end of the needle. The needle is contained within the stationary member.

In at least one aspect of the present disclosure, the movable member surrounds the stationary member. The movable member can slide distally from the stationary member. In at least one aspect of the present disclosure, the telescopic housing can further include at least one latch. When in the deployed position, the at least one latch can engage and retain a corresponding fastener to conceal the needle.

In at least one aspect of the present disclosure, the valve is a one-way valve. The one-way valve is positioned to allow a flow of fluid from the distal end of the at least one tube toward the proximal end of the hub. Flow is not permitted from the proximal end of the hub toward the distal end of the at least one tube.

In at least one aspect of the present disclosure, the hub includes a flat exterior portion. The flat exterior portion is positioned near an attachment of the at least one tube to the hub.

In at least one aspect of the present disclosure, the flat exterior portion includes an adhesive.

In at least one aspect of the present disclosure, the pneumothorax treatment device includes a grip tab. The grip tab is positioned between the hub and the telescopic housing.

In at least one aspect of the present disclosure, a pneumothorax treatment device is disclosed. The pneumothorax treatment device can include a needle including a distal end, a hub including a one-way valve, and/or a telescopic housing. The telescopic housing surrounds the needle. The telescopic housing is connected to a proximal end of the hub. The hub is further connected to at least one tube. The one-way valve is positioned to allow a flow of fluid from the distal end of the at least one tube toward the proximal end of the hub. Flow is not permitted from the proximal end of the hub toward the distal end of the at least one tube. The hub includes a flat exterior portion. The flat exterior portion is positioned near an attachment of the at least one tube to the hub. The telescopic housing can move between a deployed position and an undeployed position. In the deployed position, the distal end of the needle is concealed within the housing. In the undeployed position, the needle is unconcealed.

In at least one aspect of the present disclosure, the flat exterior portion includes an adhesive. In at least one aspect of the present disclosure, the pneumothorax treatment device includes a grip tab. The grip tab is positioned between the hub and the telescopic housing.

### BRIEF DESCRIPTION OF THE DRAWINGS

The embodiments illustrated, described, and discussed herein are illustrative of the present disclosure. As these embodiments of the present disclosure are described with reference to illustrations, various modifications or adaptations of the methods and or specific structures described may become apparent to those skilled in the art. It will be appreciated that modifications and variations are covered by the above teachings and within the scope of the appended claims without departing from the spirit and intended scope thereof. All such modifications, adaptations, or variations that rely upon the teachings of the present disclosure, and through which these teachings have advanced the art, are considered to be within the spirit and scope of the present disclosure. Hence, these descriptions and drawings should not be considered in a limiting sense, as it is understood that the present disclosure is in no way limited to only the embodiments illustrated.

FIG. 1 illustrates a side view of a pneumothorax treatment device, shown in the packaged state prior to use.

FIG. 2 illustrates a side view of a pneumothorax treatment device, shown in the packaged state prior to use.

FIG. 3 illustrates an exploded view of a pneumothorax treatment device.

FIG. 4 illustrates a side view of a pneumothorax treatment device, showing the device after the safety mechanism has been deployed and concealed the needle.

FIG. 5 illustrates a perspective view of a pneumothorax treatment device according to an embodiment of the present disclosure.

FIG. 6 illustrates a perspective of a pneumothorax treatment device according to an embodiment of the present disclosure.

### DETAILED DESCRIPTION

These descriptions are presented with sufficient details to provide an understanding of one or more particular embodiments of broader inventive subject matters. These descriptions expound upon and exemplify particular features of those particular embodiments without limiting the inventive subject matters to the explicitly described embodiments and features. Considerations in view of these descriptions will likely give rise to additional and similar embodiments and features without departing from the scope of the inventive subject matters. Although the term “step” may be expressly used or implied relating to features of processes or methods, no implication is made of any particular order or sequence among such expressed or implied steps unless an order or sequence is explicitly stated.

Any dimensions expressed or implied in the drawings and these descriptions are provided for exemplary purposes. Thus, not all embodiments within the scope of the drawings and these descriptions are made according to such exemplary dimensions. The drawings are not made necessarily to scale. Thus, not all embodiments within the scope of the drawings and these descriptions are made according to the apparent scale of the drawings with regard to relative dimensions in the drawings.

Unless defined otherwise, all technical and scientific terms used herein have the same meaning as commonly understood to one of ordinary skill in the art to which the presently disclosed subject matter pertains. Although any methods, devices, and materials similar or equivalent to those described herein can be used in the practice or testing of the presently disclosed subject matter, representative methods, devices, and materials are now described.

Following long-standing patent law convention, the terms “a,” “an,” and “the” refer to “one or more” when used in the subject specification, including the claims. Thus, for example, reference to “a device” can include a plurality of such devices, and so forth.

Unless otherwise indicated, all numbers expressing quantities of components, conditions, and so forth used in the specification and claims are to be understood as being modified in all instances by the term “about.” Accordingly, unless indicated to the contrary, the numerical parameters set forth in the instant specification and attached claims are approximations that can vary depending upon the desired properties sought to be obtained by the presently disclosed subject matter.

In some embodiments, a pneumothorax treatment device is disclosed. The pneumothorax treatment device includes a housing, a needle, a grip component, a hub, a valve, and a catheter. In some embodiments, the housing is telescopic and is operable to slide over the needle. For example, and without limitation, the telescopic housing includes at least two movable components. In some embodiments, the telescopic housing includes at least three movable components. The needle is designed to puncture a patient. Advantageously, the telescopic housing is operable to conceal the needle after the device has been positioned, thereby reducing the exposure of medical providers (e.g., physicians, nurses). In some embodiments, the hub includes a one-way valve. Advantageously this reduces the ability of air to re-enter the chest cavity of a patient. In some embodiments, the hub includes an adhesive surface operable to attach to a patient, reducing kinking or dislodgement of the device.

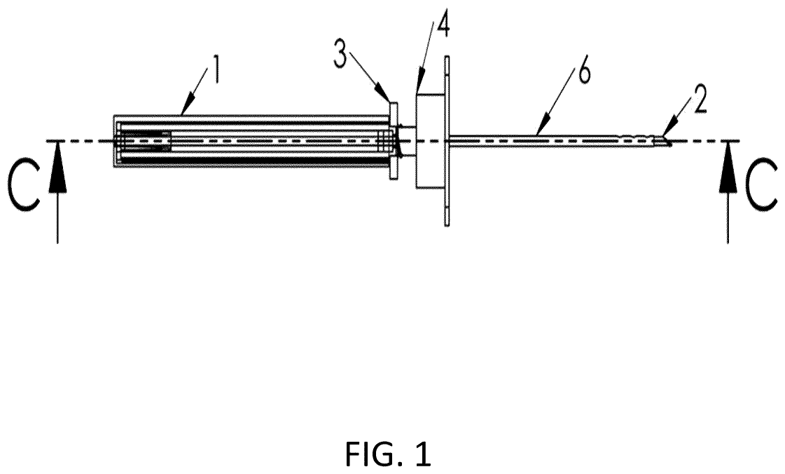

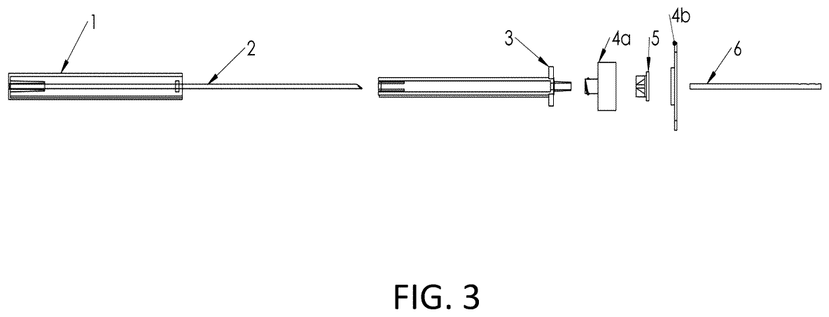

FIG. 1 illustrates a pneumothorax treatment device. The pneumothorax treatment device includes a sliding cover 1, a needle 2, a grip component 3 (e.g., finger tab), a catheter hub 4 including a valve, and a catheter 6. For example, and without limitation, the finger tab extends away from the catheter hub to enable a provider to use the finger tab as a leverage point. In some embodiments, as shown in FIG. 3, the catheter hub 4a includes a valve 5 and a locking system to connect to the telescopic system of the sliding cover system. In some embodiments, the catheter hub includes an adhesive plate 4b that sticks to a person and receives a catheter. For example, and without limitation, the adhesive place includes a double-sided adhesive. For example, and without limitation, the adhesive plate includes a medical grade adhesive (e.g., collodion). Advantageously, the adhesive plate 4b improves attachment and stability between the pneumothorax treatment device and the patient. The hub includes a Luer Lock connection. The adhesive plate includes a rigid flat surface that is perpendicular to the catheter and Luer Lock hub. The rigidness limits the bending of the catheter hub against the patient's chest, thereby maintaining perpendicularity, keeping the catheter straight. This is critical as the hub junction and catheter tubing often kinks because it sits in an unstable position against a patient's chest.

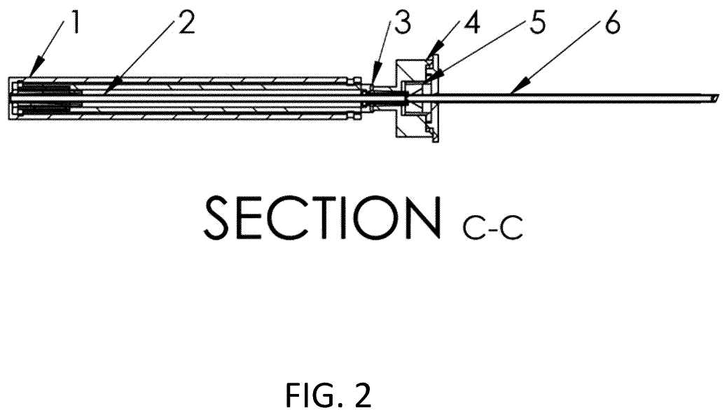

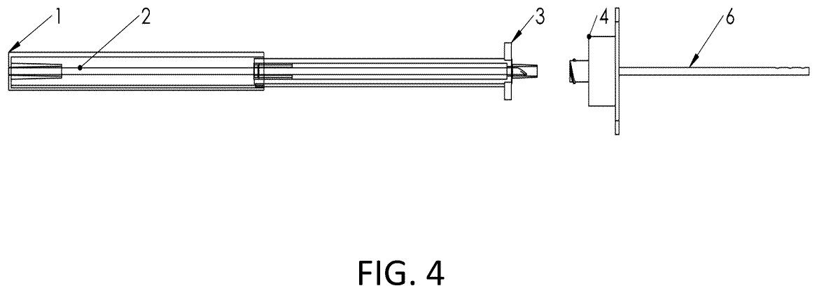

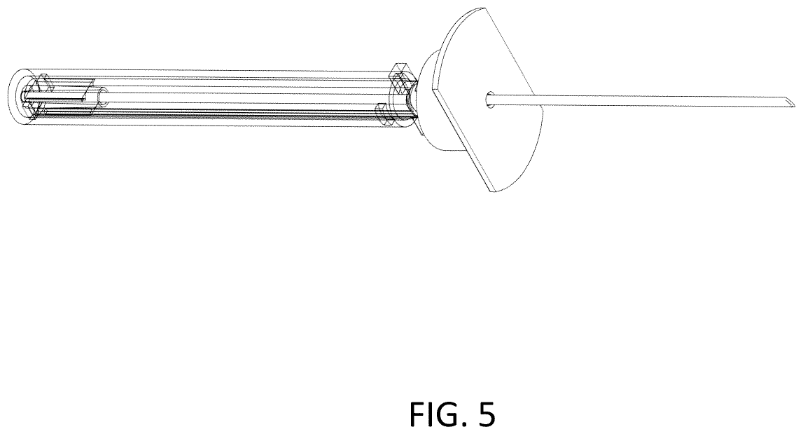

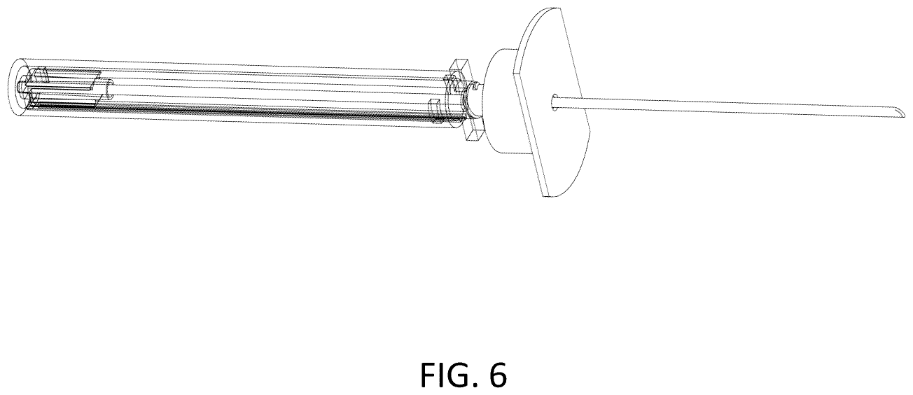

FIG. 2 illustrates a sectional view of the pneumothorax treatment device according to one embodiment of the present disclosure. FIG. 3 illustrates an exploded view of the pneumothorax treatment device. FIG. 4 illustrates a perspective view of the pneumothorax treatment device with a deployment safety mechanism to cover the needle. FIG. 5 illustrates a perspective view of a pneumothorax treatment device according to an embodiment of the present disclosure. FIG. 6 illustrates a perspective view of a pneumothorax treatment device according to an embodiment of the present disclosure.

In some embodiments, the needle is inserted into a patient's chest at a 90-degree angle relative to a patient's chest wall until the needle reaches the pleural space and air is released. Once air is released, the needle can be removed and the telescopic housing is deployed to conceal the end of the needle, thereby preventing needlestick. When deployed, the telescopic housing is locked utilizing a locking mechanism (e.g., a hook snapped into a groove). Advantageously, this prevents revealing the end of the needle after the safety mechanism is accidentally contacted.

In some embodiments, a method of using a pneumothorax treatment device is disclosed. A catheter is positioned over a needle of the pneumothorax treatment device. The needle of the pneumothorax treatment device is unsheathed using a telescopic cover system of the pneumothorax treatment device. The unsheathed needle is inserted into an intercostal space in the midclavicular line. In some embodiments, the needle is placed in the fourth or fifth intercostal space along the midaxillary line. Once the needle reaches the pleural space, the catheter is slid off of the needle and the needle is removed from a chest of a patient. In some embodiments, the patient is in a supine position. In some embodiments, when used in an emergency environment, the pneumothorax treatment device is inserted at a mid-axillary line to avoid removing a patient's vest or similar garment.

The corresponding structures, materials, acts, and equivalents of all means or step plus function elements in the claims below are intended to include any structure, material, or act for performing the function in combination with other claimed elements as specifically claimed.

Particular embodiments and features have been described with reference to the drawings. It is to be understood that these descriptions are not limited to any single embodiment or any particular set of features, and that similar embodiments and features may arise or modifications and additions may be made without departing from the scope of these descriptions and the spirit of the appended claims.

These and other changes can be made to the disclosure in light of the above Detailed Description. While the above description describes certain embodiments of the disclosure, and describes the best mode contemplated, no matter how detailed the above appears in text, the teachings can be practiced in many ways. Details of the system may vary considerably in its implementation details, while still being encompassed by the subject matter disclosed herein. As noted above, particular terminology used when describing certain features or aspects of the disclosure should not be taken to imply that the terminology is being redefined herein to be restricted to any specific characteristics, features, or aspects of the disclosure with which that terminology is associated. In general, the terms used in the following claims should not be construed to limit the disclosure to the specific embodiments disclosed in the specification, unless the above Detailed Description section explicitly defines such terms. Accordingly, the actual scope of the disclosure encompasses not only the disclosed embodiments, but also all equivalent ways of practicing or implementing the disclosure under the claims.

What is claimed is:

1. A pneumothorax treatment device comprising:
a needle including a distal end;
a hub including a valve; and
a telescopic housing;
wherein the hub is connected to a proximal end of at least one tube; and
wherein the telescopic housing is operable to move between a deployed position and an undeployed position, wherein in the deployed position, the distal end of the needle is concealed within the housing, wherein the undeployed position, the needle is unconcealed.

2. The pneumothorax treatment device of claim 1, wherein the telescopic housing includes a stationary member and a movable member, wherein the stationary member is affixed to the proximal end of the needle, wherein the needle is contained within the stationary member.

3. The pneumothorax treatment device of claim 2, wherein the movable member surrounds the stationary member, wherein the movable member is operable to slide distally from the stationary member.

4. The pneumothorax treatment device of claim 3, wherein the telescopic housing further includes at least one latch, wherein, when in the deployed position, the at least one latch is operable to engage and retain a corresponding fastener to conceal the needle.

5. The pneumothorax treatment device of claim 4, wherein the valve is a one-way valve, wherein the one-way valve is positioned to allow a flow of fluid from the distal end of the at least one tube toward the proximal end of the hub, wherein flow is not permitted from the proximal end of the hub toward the distal end of the at least one tube.

6. The pneumothorax treatment device of claim 5, wherein the hub includes a flat exterior portion, wherein the flat exterior portion is positioned near an attachment of the at least one tube to the hub.

7. The pneumothorax treatment device of claim 6, wherein the flat exterior portion includes an adhesive.

8. The pneumothorax treatment device of claim 1 further comprising a grip tab, wherein the grip tab is positioned between the hub and the telescopic housing.

9. A pneumothorax treatment device comprising:
a needle including a distal end;
a hub including a valve; and
a telescopic housing;
wherein the telescopic housing surrounds at least a portion of the needle;
wherein the telescopic housing is connected to a proximal end of the hub;
wherein the hub is further connected to at least one tube; and
wherein the telescopic housing is operable to move between a deployed position and an undeployed position, wherein in the deployed position, the distal end of the needle is concealed within the housing, wherein the undeployed position, the needle is unconcealed.

10. The pneumothorax treatment device of claim 9, wherein the telescopic housing further includes at least one latch, wherein, when in the deployed position, the at least one latch is operable to engage and retain a corresponding fastener to conceal the needle.

11. The pneumothorax treatment device of claim 9, wherein the telescopic housing includes a stationary member and a movable member, wherein the stationary member is affixed to the proximal end of the needle, wherein the needle is contained within the stationary member.

12. The pneumothorax treatment device of claim 11, wherein the movable member surrounds the stationary member, wherein the movable member is operable to slide distally from the stationary member.

13. The pneumothorax treatment device of claim 12, wherein the telescopic housing further includes at least one latch, wherein, when in the deployed position, the at least one latch is operable to engage and retain a corresponding fastener to conceal the needle.

14. The pneumothorax treatment device of claim 13, wherein the valve is a one-way valve, wherein the one-way valve is positioned to allow a flow of fluid from the distal end of the at least one tube toward the proximal end of the hub, wherein flow is not permitted from the proximal end of the hub toward the distal end of the at least one tube.

15. The pneumothorax treatment device of claim 14, wherein the hub includes a flat exterior portion, wherein the flat exterior portion is positioned near an attachment of the at least one tube to the hub.

16. The pneumothorax treatment device of claim 15, wherein the flat exterior portion includes an adhesive.

17. The pneumothorax treatment device of claim 9 further comprising a grip tab, wherein the grip tab is positioned between the hub and the telescopic housing.

18. A pneumothorax treatment device comprising:
a needle including a distal end;
a hub including a one-way valve; and
a telescopic housing;
wherein the telescopic housing surrounds the needle;
wherein the telescopic housing is connected to a proximal end of the hub;
wherein the hub is further connected to at least one tube;
wherein the one-way valve is positioned to allow a flow of fluid from the distal end of the at least one tube toward the proximal end of the hub, wherein flow is not permitted from the proximal end of the hub toward the distal end of the at least one tube;
wherein the hub includes a flat exterior portion, wherein the flat exterior portion is positioned near an attachment of the at least one tube to the hub; and
wherein the telescopic housing is operable to move between a deployed position and an undeployed position, wherein in the deployed position, the distal end of the needle is concealed within the housing, wherein the undeployed position, the needle is unconcealed.

19. The pneumothorax treatment device of claim 18, wherein the flat exterior portion includes an adhesive.

20. The pneumothorax treatment device of claim 19 further comprising a grip tab, wherein the grip tab is positioned between the hub and the telescopic housing.
